(19) United States
(12) Patent Application Publication
(10) Pub. No.: US 2022/0096260 A1
(43) Pub. Date: Mar. 31, 2022

(54) PELVIC TILT BRACE

(71) Applicant: BraceAbility, Inc., Cedar Falls, IA (US)

(72) Inventor: Melanie Deborah Hatto, Cape Coral, FL (US)

(21) Appl. No.: 17/035,189

(22) Filed: Sep. 28, 2020

(51) Int. Cl. A61F 5/02

(52) U.S. Cl. CPC A61F 5/028

(57) ABSTRACT

A pelvic tilt brace includes a main structural member for wrapping around a waist of a wearer including an inner and an outer side, and a fastening mechanism having a first and a second securing feature on opposite ends of the main structural member for fastening together. A first semi-rigid component pair and a second semi-rigid component pair that each include front and back components positioned opposite one another on the outer side, each including cord-involving hardware thereon. Cord portions are between the front and back components for the semi-rigid component pairs, with ≥1 cord attachment having a cord attachment end attached to the cord portions. The pelvic tilt brace is configured so that after being secured to the waist, and the cord attachment end is pulled forward and then secured, the semi-rigid component pairs apply a pressure to the pelvis providing a more neutral condition for the pelvis.

### FIELD

This Disclosure relates to braces for the pelvic area and the adjacent portion of the lower back including the hips of a wearer.

### BACKGROUND

The pelvis (the plural generally called the pelves or pelvises) is either the lower part of the trunk of the human body between the abdomen and the thighs or the skeleton embedded in it, sometimes also called bony pelvis or pelvic skeleton. The pelvic region of the trunk includes the bony pelvis, the pelvic cavity which is the space enclosed by the bony pelvis, the pelvic floor, below the pelvic cavity, and the perineum, below the pelvic floor. The pelvic skeleton is formed in the area of the back, by the sacrum and the coccyx and anteriorly and to the left and right sides, by a pair of hip bones.

The two hip bones connect the spine with the lower limbs. The hip bones are attached to the sacrum posteriorly, connected to each other anteriorly, and joined with the two femurs at the hip joints. The gap enclosed by the bony pelvis, called the pelvic cavity, is the section of the body underneath the abdomen and includes the reproductive organs (sex organs) and the rectum, while the pelvic floor at the base of the cavity assists in supporting the organs of the abdomen.

Anterior pelvic tilt is a change in posture of an individual that happens when the front of their pelvis rotates forward, and the back of their pelvis rises. Anterior pelvic tilt is present when the anterior superior iliac spine (ASIS, also known as hip points) is tipped forward of the pubic bone in the coronal plane. The coronal plane as conventionally defined divides an individual in half creating a front (anterior) portion and a back (posterior) portion. Anterior pelvic tilt is known to be caused by a muscle imbalance often related to excessive time sitting or the lack of physical activity that affects one's posture and the shape of their spine, which can lead to other symptoms.

The term “posture” is generally defined as a composite of the positions of all the joints of the human body at any given moment. An ideal posture is stable, where postural alignment maintains the body's mass over its base of support to minimize stress and strain on tissues statically, at rest dynamically, and during movement which minimizes energy cost.

In the case of anterior pelvic tilt, the pelvis can generally be gradually returned to a neutral position meaning the position of the pelvis where the ASIS and pubic bone are in line with each other in the coronal plane vertical while standing, or horizontal when lying down, using a variety of stretching and strengthening exercises. These exercises are known to include squats and the pelvic tilt. A neutral pelvis is important because it allows for a neutral spine which allows for the maximum amount of space between each vertebra as they are aligned in their natural curves.

### SUMMARY

This Summary is provided to introduce a brief selection of disclosed concepts in a simplified form that are further described below in the Detailed Description including the drawings provided. This Summary is not intended to limit the claimed subject matter's scope.

The problem of anterior pelvic tilt is solved by disclosed pelvic tilt braces that are adjustable in the amount of applied pressure to the wearer responsive to a wearer's pressure settings, which rotate and straighten the ASIS with respect to the pubic bone of the wearer to provide a neutral position so that they are aligned in the coronal plane. Disclosed pelvic tilt braces perform this function by applying a pressure that forces the front of the hips in a compressive manner backward which changes the angle of the anterior tilted hip bones in the backward direction so that it results in a more neutral pelvis. In a more neutral pelvis position, when viewed from the side of the wearer, the pelvis' left side is at essentially the same level as its right side.

One disclosed embodiment comprises a pelvic tilt brace including a main structural member that has a sufficient length to wrap around a waist of a wearer comprising an inner and an outer side, and a fastening mechanism having a first securing feature and a second securing feature (such as a hook side and a loop side) on opposite ends of the main structural member for fastening together. A first semi-rigid component pair and a second semi-rigid component pair each include a front and a back component positioned opposite one another on the outer side, each of the first and second semirigid component pairs including cord-involving hardware thereon.

Cord portions are between the front and back components for each of the first and the second semi-rigid component pair, with at least one cord attachment having a cord attachment end attached to the cord portions. The pelvic tilt brace is configured so that after being secured to the waist, and the cord attachment end is pulled forward and is then secured, the first and second semi-rigid component pairs apply a pressure to the pelvis of the wearer that results in a more neutral condition for the pelvis.

### BRIEF DESCRIPTION OF THE DRAWINGS

FIG. 1A is a front view depiction of a wearer having a disclosed pelvic tilt brace positioned around his or her waist, before pulling the cord attachment ends forward then securing the cord attachment ends generally to the main structural member, according to an example embodiment.

FIG. 1B is a front view depiction of a wearer holding a disclosed pelvic tilt brace before the wearer completes wrapping of the pelvic tilt brace around his or her waist, showing one side (such as the hook side or the loop side) of the fastening mechanism, according to an example embodiment.

FIG. 2 is a side view depiction of the left side of a wearer having a disclosed pelvic tilt brace positioned around their waist, before securing the cord attachment ends that are in the hands of the wearer, according to an example embodiment.

FIG. 3 is a posterior view depiction of a wearer having a disclosed pelvic tilt brace secured around his or her waist, according to an example embodiment.

FIG. 4A shows a frontal view with known components of a pelvis identified including the ASIS, posterior superior iliac spine (PSIS), and the pubic bone including its furthest edge. FIGS. 4B and 4C are side view depictions showing the pelvis representing the pelvis before and after wearing a disclosed pelvic tilt brace, as an anterior pelvic tilt condition, and a neutral pelvis condition, respectively.

### DETAILED DESCRIPTION

Disclosed embodiments are described with reference to the attached figures, wherein like reference numerals are used throughout the figures to designate similar or equivalent elements. The figures are not drawn to scale and they are provided merely to illustrate certain disclosed aspects. Several disclosed aspects are described below with reference to example applications for illustration. It should be understood that numerous specific details, relationships, and methods are set forth to provide a full understanding of the disclosed embodiments.

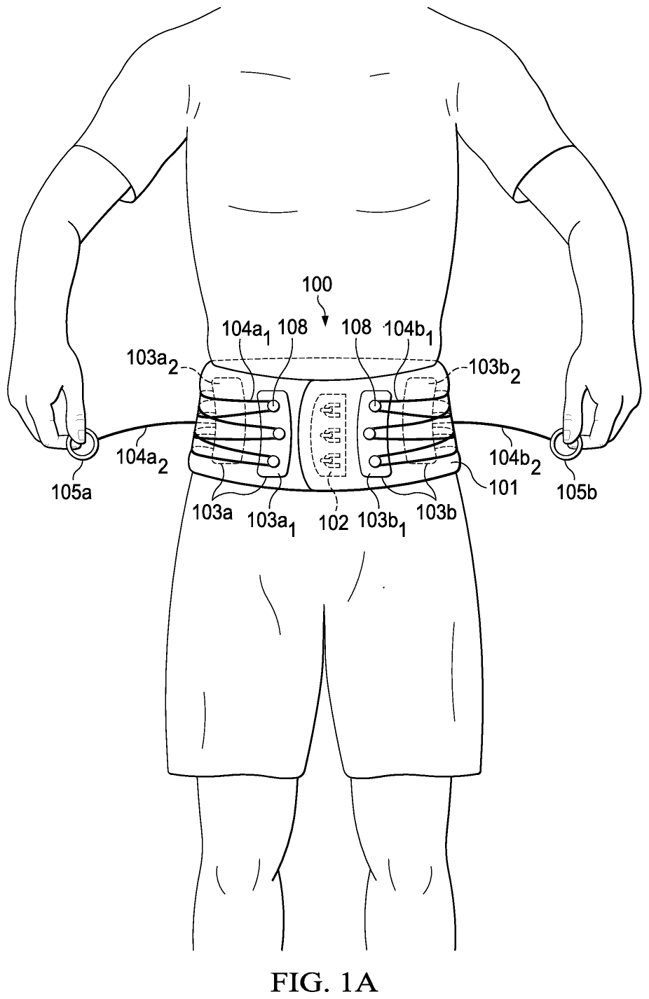

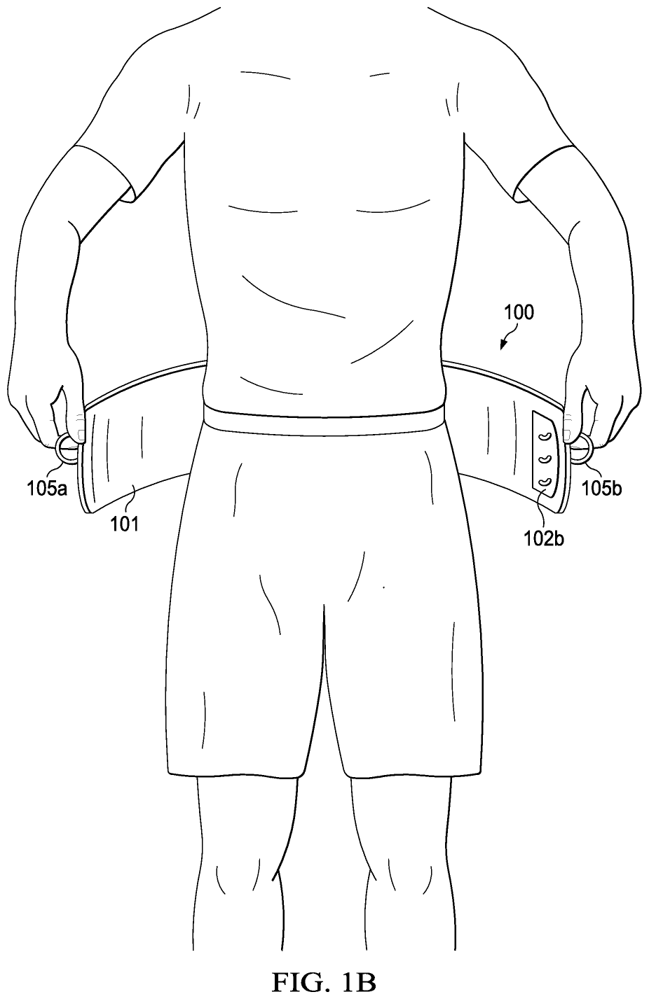

FIG. 1A is a front view depiction of a wearer having a disclosed pelvic tilt brace 100 positioned around his or her waist, before pulling the respective cord attachment ends 105a, 105b forward then securing the cord attachment ends, generally being secured to the main structural member 101, according to an example embodiment. Besides the cord attachment ends 105a, 105b being secured to the main structural member 101, the cord attachment ends 105a, 105b can also be positioned through a sleeve for securing, which can be in the attachment zone in the front of the wearer.

The main structural member 101 typically comprises an elastomeric material, such as neoprene, has a sufficient length to wrap around a waist of the wearer, and includes an inner side and an outer side. There is a fastening mechanism shown as 102 having a first securing feature on one end of the main structural member 101 and a second securing feature on an opposite end of the main structural member 101 for fastening together. Neoprene is known to be a family of synthetic rubbers produced by polymerization of chloroprene, where neoprene is known to exhibit good chemical stability and it maintains flexibility over a wide temperature range.

One side of the inner side of the main structural member 101 attaches to the opposite outer side of the main structural member 101 in the front of the pelvic tilt brace 100, generally with a hook-based fastener arrangement, such as comprising a hook and loop arrangement, for example using VELCRO or a similar fastening structure. The outer layer of the main structural member 101 may be a different material attached (e.g., the attachment being sewn stitches, encased in a sleeve, or attached by an adhesive) to the outer side of the main structural member 101. The main structural member 101 can itself provide the outer layer.

The pelvic tilt brace 100 includes on its outer side a first semi-rigid component pair 103a including a front component 103a₁ and a back component 103a₂ (enhanced view to show otherwise non-visible in the view provided of the back semi-rigid component 103a₂) shown on the right of the wearer, and a second semi-rigid component pair 103b including a front component 103b₁ and a back component pair 103b₂ (enhanced view to show otherwise non-visible in the view provided of the second back component 103b₂) shown on the left of the wearer. The respective semi-rigid component pairs 103a and 103b are positioned opposite one another on the outer side of the main structural member 101 each including a plurality of cord-involving hardware 108 thereon, such as buckles and/or pulleys.

The front components 103a₁ and 103b₁ of each semi-rigid component pair 103a and 103b are positioned to be in front of the wearer's ASIS, which is the bony prominence at the front of one's waist, and the back components 103a₂ and 103b₂ of each of the semi-rigid component pairs 103a and 103b are positioned to be over the wearer's PSIS. After the main structural member 101 is attached, the wearer may adjust the position of the pelvic tilt brace 100 to position the respective semi-rigid components in these intended places by rotating the pelvic tilt brace 100 along their waistline.

Regarding the term ‘semi-rigid components’, rigidity, also known as “stiffness,” is generally measured using Young's modulus that is also called an elastic modulus. The Young's modulus can be defined as the force necessary to bend a material to a given degree. The room temperature Young's modulus/elastic modulus values for the components 103a₁, 103a₂ and 103b₁, 103b₂ can be 0.05 to 5.0 GPa. Acrylonitrile butadiene styrene (ABS), polypropylene, high-density polyethylene (HDPE), and neoprene rubber, are example semi-rigid materials that generally may be used for the components 103a₁, 103a₂ and 103b₁, 103b₂ for the semi-rigid component pair 103a and 103b of disclosed pelvic tilt braces.

A pulley as used herein and as known is a wheel on an axle or shaft that is designed to support movement and change of direction of a taut rope, cable or belt, or transfer of power between the shaft and the rope, cable or belt. A buckle (also called a clasp) is a device used for fastening two loose ends, with one end attached to it and the other held by a catch in a secure but adjustable manner.

There are cord portions between the front and back components of each semi-rigid component pair shown as 104a₁ for the semi-rigid component pair 103a on the right side and 104b₁ semi-rigid component pair 103b on the left side. Disclosed cords can utilize an elastic material such as nylon woven, or a non-elastic material such as waxed or braiding cord. There is also movable cord portion 104a₂ on the right side between the cord portion 104a₁ and the cord attachment end 105a, and a movable cord portion 104b₂ on the left side between the cord portion 104b₁ and the cord attachment end 105b.

The movable cord portions 104a₂ and 104b₂ permit the above described forward motion that reduces the distance between the front components 103a₁, 103b₁ and back components 103a₂ and 103b₂ of each semi-rigid component pair 103a, 103b which applies a pressure to the pelvis resulting in anterior lifting and pulling that provides a more neutral condition for the pelvis, thus providing better alignment of the ASIS and the pubic bone to be in line with each other in the frontal plane. This advantageously changes the wearer from being in an anteriorly tilted pelvis position into an essentially neutral pelvis position.

The pelvic tilt brace 100 is generally provided in a variety of different lengths, such as a smaller length for smaller/thin people, a medium length for medium people, and a longer length for larger/overweight people. The width (or height) of the main structural member 101 generally is varied less as compared to its length. For example, the width of the pelvic tilt brace 100 is generally between 3 inches to 10 inches and the length can generally range from 25 inches up to about 60 inches.

FIG. 1B is a front view depiction of a wearer holding the respective cord attachment ends 105a, 105b of a disclosed pelvic tilt brace 100 before the wearer completes wrapping of the main structural member 101 the pelvic tilt brace 100 around his or her waist, showing the one side (such as the hook side or the loop side) of the fastening mechanism for the main structural member 101 shown as 102b on the inner side of the main structural member 101, according to an example embodiment. Assuming the fastening mechanism 102b comprises hooks, the fastening mechanism on the inner side of the main structural member 101 may comprise loops, typically being on the outer side of the main structural member 101.

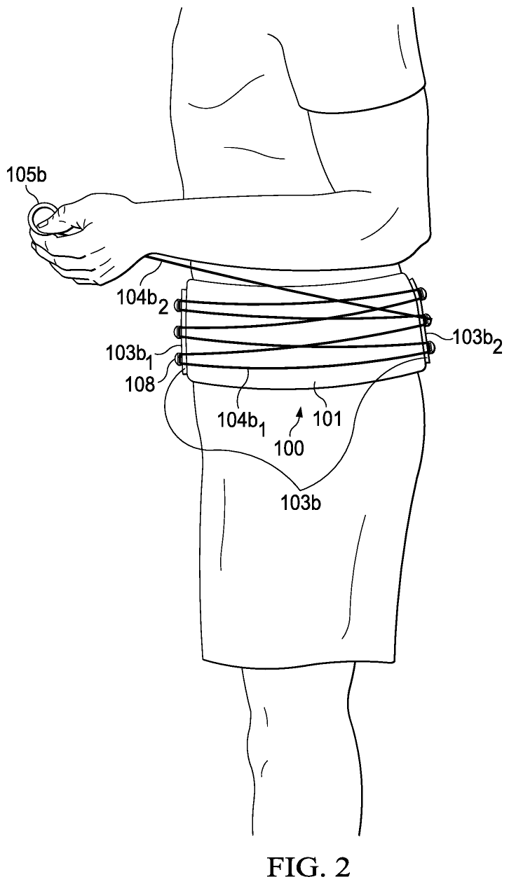

FIG. 2 is a side view depiction of the left side of a wearer having a disclosed pelvic tilt brace 100 positioned around his or her waist, before securing the cord attachment ends shown as 105b in the hand of the wearer to the main structural member 101, according to an example embodiment. The cord portion 104b₁ connects the cord-involving hardware 108 on the respective components on each side of the wearer, shown in FIG. 2 connecting the front component 103b₁ to the back component 103b₂ of the semi-rigid component pair 103b.

The wearer pulls and then secures the cord attachment ends shown as cord attachment end 105b which pulls the respective cords shown as cord portion 104b₂ forward to have the semi-rigid component pairs shown as the semi-rigid component pair 103b apply pressure that compresses and rotates the wearer's hips posteriorly into frontal alignment. The wearer can know how much tension to create before securing the cord attachment end by feeling their pelvis rotate up and back, and their back/spine elongate, so that the force being applied will hold the pelvis in this more neutral position. By virtue of the wearer pulling the respective cord attachment ends shown in FIG. 2 as 105b and then securing them, pressure is applied through the cord portions shown in FIG. 2 as 104b₁ onto the semi-rigid component pairs to the main structural member 101 in the front and side of the wearer, resulting in tilting the pelvic area of the wearer backward to provide an essentially neutral pelvis.

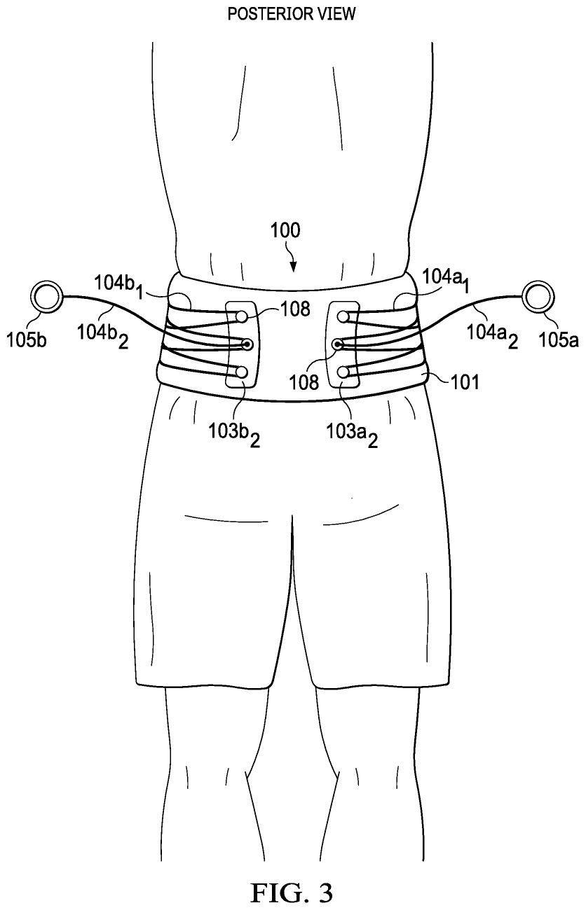

FIG. 3 is a posterior view depiction of a wearer having a disclosed pelvic tilt brace 100 secured around his or her waist, according to an example embodiment. The back components 103a₂ and 103b₂ are shown. As noted above, the back components 103a₂ and 103b₂ are positioned over the wearer's PSIS located in the back portion of the wearer's pelvis.

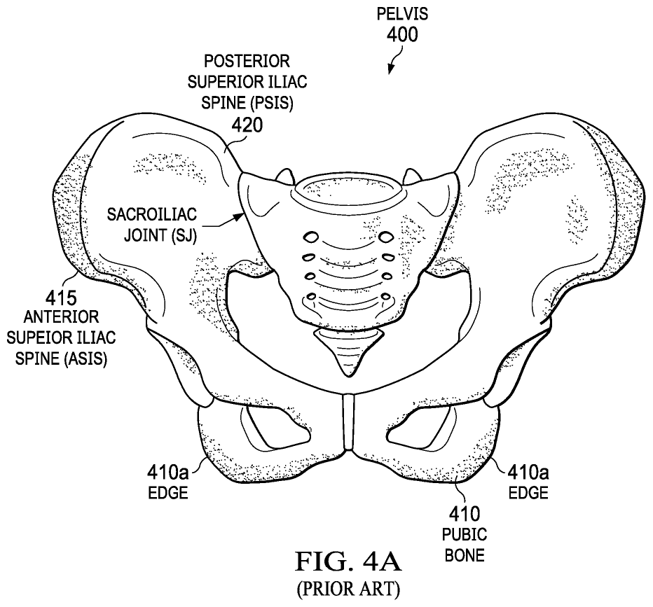

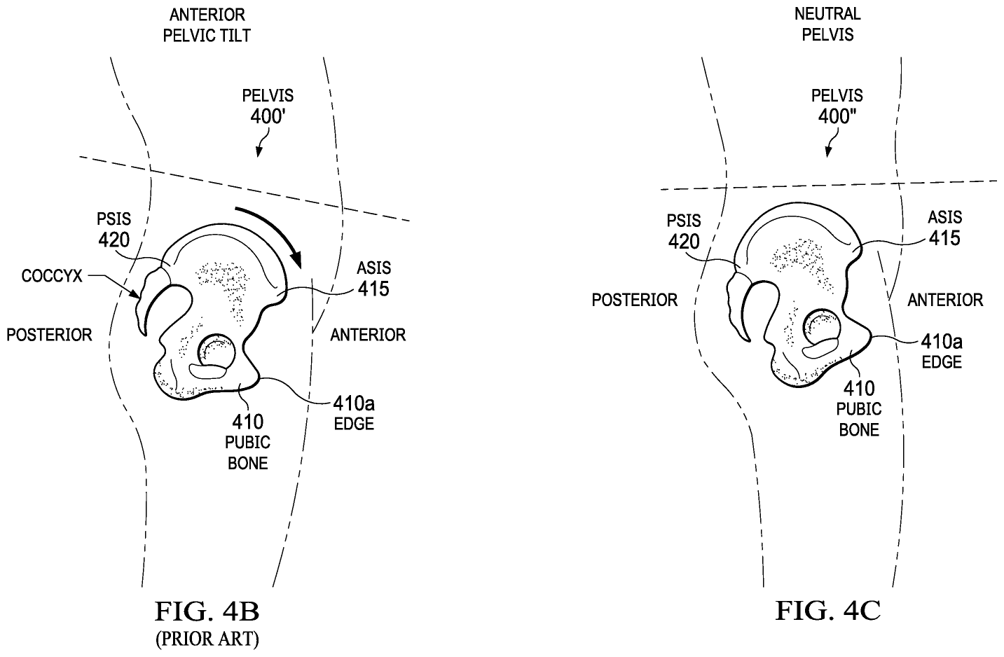

FIG. 4A shows a frontal view with some known components of a pelvis 400 identified including the ASIS 415, posterior superior iliac spine (PSIS) 420, and the pubic bone 410 including its furthest edge 410a. FIGS. 4B and 4C are side view depictions showing the pelvis as 400′ and 400″ respectively, representing the pelvis before and after wearing a disclosed pelvic tilt brace. The pelvis 400′ shown in FIG. 4B as an anterior pelvic tilt evidences an anterior pelvic tilt condition, where the ASIS 415 can be seen to be rotated forward relative to the furthest edge 410a of the pubic bone 410, with the arrow provided in FIG. 4B showing the angle and direction of the forward hip rotation which results in the ASIS 415 being forward relative to the furthest edge 410a of the pubic bone 410. The dashed line shown in FIGS. 4B and 4C provides a reference to indicate the measurement of pelvic tilt in degree from the relation of the PSIS to the ASIS 415. If the pelvis is rotated forward to a point, then the spine will compensate by creating a lordotic curve to keep the person's head in a vertically upright position. Pelvic rotation can also create tight quadriceps muscles which result in knee injuries.

FIG. 4C shows the pelvis as a neutral pelvis 400″ provided while wearing a properly secured disclosed pelvic tilt brace (brace not shown to avoid obscuring pelvic features) featuring a frontal plane alignment condition, where the ASIS 415 and the furthest edge 410a of the pubic bone 410 are in a line with each other, where the line is essentially parallel to a frontal plane of the wearer. As used herein, the term ‘essentially parallel’ means±5° relative to a frontal plane of the wearer. Disclosed pelvic tilt braces 100 solve problems associated with pelvic rotation, including the above-described problems.

Disclosed pelvic tilt braces 100 can be worn over or worn under a wearer's clothing. The main structural member 101 of the pelvic tilt brace 100 is worn at the waistline of the wearer. The center of the inside surface of the pelvic tilt brace 100 is placed over the wearer's lumbo-sacrum spine.

Disclosed pelvic tilt braces can be applied to generally a variety of different medical conditions and discomforts that can each benefit from providing support for over lengthened/stretched hip extensors while straightening/stretching the tight hip flexors. For example, medical conditions addressed by disclosed pelvic tilt braces include but are not limited to, anterior pelvic tilt, lower cross syndrome, lordosis, iliopsoas syndrome, hip flexor conditions, pregnancy, and pronounced lumbar curve, lordosis. Disclosed pelvic tilt braces can be worn during activities, or while the wearer is at rest including when the wearer is in a seated position.

While various disclosed embodiments have been described above, it should be understood that they have been presented by way of example only, and not limitation. Numerous changes to the subject matter disclosed herein can be made in accordance with this Disclosure without departing from the spirit or scope of this Disclosure. For example, although disclosed pelvic tilt braces are generally described having one cord associated with each semi-rigid component pair, it may be possible to use a single cord by redesigning the components. In addition, while a particular feature may have been disclosed with respect to only one of several implementations, such feature may be combined with one or more other features of the other implementations as may be desired and advantageous for any given or particular application.

1. A method aligning a pelvis of a wearer, comprising:
providing a pelvic tilt brace consisting of a one piece main structural member that has a width and a sufficient length to wrap around a waist of a wearer having an inner side and an outer side, a fastening mechanism having a first securing feature on one end of the main structural member and a second securing feature on an opposite end of the main structural member for fastening together, each of the first and the second semi-rigid component pairs each including a plurality of cord-involving hardware thereon oriented along the width direction, with cord portions between the front and the back components of each of the first and the second semi-rigid component pairs;
the wearer:
wrapping the pelvic tilt brace across the waist so that the front components are positioned on a front portion of the pelvis and the back components are positioned on a back portion of the pelvis;
fastening the first securing feature to the second securing feature;
pulling at least one cord attachment having a cord attachment end coupled to the cord portions forward, and
after the pulling, securing the cord attachment end,
wherein the first and the second semi-rigid component pairs each apply a pressure to the pelvis that results in a more neutral condition for the pelvis.

2. The method of claim 1, wherein the front components are positioned over an anterior superior iliac spine (ASIS) of the wearer in the front portion of the pelvis, and wherein the back components are positioned over a posterior superior iliac spine (PSIS) of the wearer in the back portion of the pelvis.

3. The method of claim 2, wherein the more neutral condition for the pelvis comprises alignment of the ASIS and a furthest edge of a pubic bone to be in a line with each other that is within ±5° relative to a frontal plane of the wearer.

4. The method of claim 1, wherein the main structural member comprises an elastomeric material, and wherein the front and the back components comprise a polymer having a Young's modulus at room temperature between 0.05 GPa and 5.0 GPa.

5. The method of claim 1, wherein the first and the second semi-rigid component pairs both extend in a direction of the width spanning a majority of the width.

6. The method of claim 1, wherein the cord-involving hardware comprises at least one of buckles and pulleys.

7. The method of claim 1, wherein the first securing feature and the second securing feature together provide a hook and loop fastener.

8. The method of claim 1, wherein the pulling at least one cord attachment comprises pulling a first cord having a first cord attachment end and a second cord having a second cord attachment end.

9. A pelvic tilt brace, comprising:
only a one piece main structural member that has a width and a sufficient length to wrap around a waist of a wearer comprising having an inner side and an outer side,
a fastening mechanism having a first securing feature on one end of the main structural member and a second securing feature on an opposite end of the main structural member for fastening together,
a first semi-rigid component pair including a front and a back component and a second semi-rigid component pair including a front and a back component positioned opposite one another on the outer side, each of the first and the second semi-rigid component pairs each including a plurality of cord-involving hardware thereon oriented along the width direction;
cord portions between the front and back components for the first and second semi-rigid component pairs, with at least one cord attachment having a cord attachment end attached to the cord portions;
wherein when the pelvic tilt brace is configured so that after being secured to the waist, and the cord attachment end is pulled forward and then secured, the first and the second semi-rigid component pairs apply a pressure to a pelvis of the wearer that results in a more neutral condition for the pelvis.

10. The pelvic tilt brace of claim 9, wherein the first and the second semi-rigid component pairs are configured after the being secured so that the front components are positioned over a user's anterior superior iliac spine (ASIS) in a front portion of the pelvis, and wherein the back components are positioned to be over a sacroiliac joint in a back portion of the pelvis.

11. The pelvic tilt brace of claim 9, wherein the cord portions comprise a first cord portion associated with the first semi-rigid component pair and a second cord portion associated with the second semi-rigid component pair, and wherein the at least one cord attachment end comprises a first cord attachment end coupled to the first cord portion and a second cord attachment end coupled to the second cord portion.

12. The pelvic tilt brace of claim 9, wherein the main structural member comprises an elastomeric material, and wherein the front and back components comprise a polymer having a Young's modulus at room temperature between 0.05 GPa and 5.0 GPa.

13. The pelvic tilt brace of claim 9, wherein the cord-involving hardware comprises at least one of buckles and pulleys.

14. The pelvic tilt brace of claim 9, wherein the first and the second semi-rigid component pairs both extend in a direction of the width spanning a majority of the width.

15. The pelvic tilt brace of claim 9, wherein the front components are configured to be positioned over an anterior superior iliac spine (ASIS) in a front portion of the pelvis, and wherein the back components are positioned over a posterior superior iliac spine (PSIS) in a back portion of the pelvis.

16. The pelvic tilt brace of claim 9, wherein the first securing feature and the second securing feature together provide a hook and loop fastener.
